(19) United States
(12) Patent Application Publication
(10) Pub. No.: US 2015/0374660 A1
(43) Pub. Date: Dec. 31, 2015

(54) Ascorbate Esters of Omega-3 Fatty Acids and Their Formulations and Uses

(71) Applicant: JIVA PHARMA, INC., Ann Arbor, MI (US)

(72) Inventor: Om P Goel, Ann Arbor, MI (US)

(73) Assignee: JIVA PHARMA, INC., Ann Arbor, MI (US)

(21) Appl. No.: 14/769,673

(22) PCT Filed: Feb. 25, 2014
(86) PCT No.: PCT/US2014/018353

§ 371 Date: Aug. 21, 2015

Related U.S. Application Data
(60) Provisional application No. 61/769,326, filed on Feb. 26, 2013.

(51) Int. Cl. A61K 31/375; A61K 31/155; A61K 45/06; C07D 307/62

(52) U.S. Cl. CPC A61K 31/375; C07D 307/62; A61K 31/155; A61K 45/06

(57) ABSTRACT

The present invention provides ascorbate esters of omega-3 fatty acids as their pharmaceutically-acceptable salts and pharmaceutically-acceptable formulations for use as dietary supplements with enhanced antioxidant properties, in improving the effectiveness of other drugs and in treatment of hypertriglyceridemia, hypercholestolemia, diabetes, psychiatric and neurological disorders, attention deficit and hyperactivity disorder, and as cosmeceuticals.

### FIELD OF THE INVENTION

The present invention concerns ascorbate esters of omega-3 fatty acids and their formulations and use in treating various medical conditions such as hypertriglyceridemia, cardiovascular disease, diabetes, psychiatric and neurological disorders, attention deficit and hyperactivity disorder, and Alzheimer's disease, as well as their use as neutraceuticals and cosmeceuticals.

### BACKGROUND OF THE INVENTION

### Omega-3 Oils or Omega-3 Fatty Acids

Omega-3 oils or omega-3 fatty acids are naturally occurring, straight-chain (16-24 carbons) fatty carboxylic acids, that are essential for normal metabolism in humans and other animals. These omega-3 fatty acids are not synthesized by the human body, and are recommended to be taken as dietary supplements in 1-4 grams daily. The health benefits of omega-3 fatty acids in fighting cancer, cardiovascular disease, dyslipidemia, stroke, inflammation, developmental disorders in children, cognitive aging, and psychiatric disorders are the subject of numerous clinical studies (Wikipedia). There is rapid growth in the omega-3 acids' consumer market for neutraceuticals; and this is expected to continue at a robust pace for at least the near term.

Nordic Naturals markets even a baby omega-3 supplement (Baby's DHA) that claims to help support learning and language abilities, and healthier development of brain and eyes. Clinical studies in 4 year old children support the beneficial effects of docohexaenoic acid (DHA) on cognitive function (NCT 00351624; 2006-2008; sponsored by Martek BioSciences Corporation). It would be an interesting study to follow such treated children over decades regarding the incidence of onset of symptoms of Alzheimer's disease relative to the untreated group.

The Food and Nutrition Board in the United States in 2002 has established recommended Adequate Intakes (AI values) of omega-3 fatty acids ALA, DHA and EPA for infants, children and adults. These values are readily accessible.

Currently, attention is drawn by NCT 00351624; 2006-2008 (sponsored by Martek BioSciences Corporation) to the possible benefits of omega-3 supplements in treating school children of 6-12 years age, as well as teenagers, who are diagnosed with symptoms of attention deficit hyperactivity disorder (ADHD). Prescribed treatments for ADHD are various stimulant medications, such as atomoxetine hydrochloride (Concerta®, trademark of ALZA Corporation), methylphenidate (Ritalin®, trademark of NOVARTIS CORPORATION), dextroamphetamine plus amphetamines (Adderall®, trademark of SHIRE LLC) and lisdexamfetamine (Vynanse®, trademark of SHIRE LLC), and others.

These omega-3 fatty acids have 3-6 conjugated carbon-carbon double bonds, and are so named as the first carbon with unsaturation is 3^{rd} carbon from the distal carboxylic acid carbon. All double bonds are in the cis configuration.

Among these omega-3 fatty acids, eicosapentanenoic acid (EPA, 20 carbons, 5 conjugated carbon-carbon double bonds), docohexaenoic acid (DHA, 22 carbons, 6 double bonds), and α-linolenic acid (ALA, 18 carbons, 3 double bonds), are the most studied omega-3 fatty acids pharmacologically. The two most important omega-3 fatty acids are EPA and DHA, which are found most abundantly in marine sources such as fish oils from salmon, mackerel, sardines, herring, anchovies, krill, squid, greenshell/lipped mussels, and algae oil. ALA is abundant in walnuts, flaxseed (55%), soybean oil, and green vegetables such as spinach, kale, Brussels sprouts and similar vegetables. Most people get sufficient ALA needed from their regular diets.

Great strides have been made in developing and producing vegetarian omega-3s from novel genetic strains of algae. Vegetarian docosahexaenoic acid (DHA) products are made by fermentation of novel algal strains, now available from various groups, for example, from the Lonza Group. Perhaps, this is the reason for wide spread distribution of DHA in numerous products in the groceries' aisles, including milk and dairy. DSM Nutritional Products has introduced life's DHA plus EPA together in a vegetarian format made entirely from algae. In the future, vegetarian sources of omega-3s, produced under a controlled environment, would offer abundant quantities of important omega-3 oils which are free of environmental pollutants (discussed below).

The chemical synthesis of omega-3 fatty acids is long, expensive, and not practical for producing commercial quantities.

A major drawback of using the plentiful marine sources of omega-3 fatty acids is the levels of toxic environmental pollutants in the crude oils removed from the fish and algae. These include polychlorinated biphenyls (PCBs), polybrominated biphenyls (PBBs), dioxins, dichlorodiphenyltrichloroethane (DDT), dieldrin, polychlorinated benzofurans, and heavy metals such as mercury, lead, tin, and arsenic as their organic derivatives such as methyl mercury. Elegant, extensive industrial purification methods have been developed to reduce environmental pollutants to sub-toxic levels, and produce large amounts of omega-3 oils for pharmacology and clinical development (e.g., U.S. Pat. No. 7,732,488). By the processes described, pharmaceutically effective mixtures of ethyl esters of eicosapentaenoic acid and docosahexaneoic acid are produced to treat hypertriglyceridemia. For example, the drug Lovaza™ (developed by Reliant Pharmaceuticals and marketed by GlaxoSmithKline (GSK)) is approved by the US FDA to lower very high triglyceride levels ≧500 mg/dl. Lovaza™ is supplied as a liquid-filled gelatin capsule for oral administration. Each 1-gram capsule contains at least 900 mg of the ethyl esters of six different omega-3 fatty acids sourced from fish oils. These are predominantly a combination of ethyl esters of eicosapentaenoic acid (EPA—approximately 465 mg) and docosahexaenoic acid (DHA—approximately 375 mg). These ethyl esters act as prodrugs and are metabolized to active free acids. Lovaza™ lowers VLDL-cholesterol, and raises HDL-cholesterol. However, it can raise LDL-cholesterol up to 45% as reported in Pharmacy & Therapeutics (May 2008) “Omega-3-acid Ethyl Esters (Lovaza) For Severe Hypertriglyceridemia”.

Interestingly, Amarin Corporation studied highly purified EPA ethyl ester, with ≦3% levels of the DHA ethyl ester component, and found that it also lowers triglycerides in patients with ≧500 mg/dl, yet does not raise, but lowers LDL-C, unlike Lovaza™. In July 2012, the US FDA approved Amarin's Vascepa™ (icosapent ethyl, EPA ethyl ester) for treating severe hypertriglyceridemia (U.S. Pat. No. 8,188,146). Results from a Japanese clinical study suggest that the addition of EPA ethyl ester to statin therapy prevents major coronary events, angina pectoris and clinical myocardial infarctions (Wikipedia). However, the US FDA has not approved Vascepa™ to reduce the risk of cardiovascular disease, and further long term clinical studies have been requested.

U.S. Pat. No. 8,188,146 also offers clinical evidence of pure ethyl-EPA to be effective in the treatment of psychiatric and neurological disorders such as depression, schizophrenia, bipolar disorder, Huntington's disease and others when used in combination with standard therapies.

The mechanisms by which omega-3 fatty acids lower circulating triglycerides are being actively studied. One theory is that the omega-3 fatty acids inhibit the formation of VLDL in the liver, which in turn lowers the level of circulating triglycerides. It is possible that they act through similar cellular pathways of lipid and lipoprotein metabolism, and induction of the beta-oxidation pathway, as fibric acids, such as benzfibrate, fenofibrate and gemfibrozil. However, unlike the fibrates, which are peroxisome proliferator-activator receptor alpha (PPARα) agonists, the omega-3 acids DHA and EPA are PPARγ activators. Both receptors have a distinct tissue expression; PPARα is expressed at high levels in the liver, whereas PPARγ is expressed in many tissues, with the highest concentrations in adipose tissue. PPARγ agonists pioglitazone and omega-3 fatty acids have been shown to stimulate adiponectin production; A. Banga et al., Am J Physiol. Endocrinol. Metab. 296(3), 13-14 (March 2009). This difference is very significant, as discussed later in this specification).

### Type2 Diabetes

Type2 diabetes (T2D) is a life-long, disease of elevated plasma glucose levels caused by a combination of peripheral insulin resistance (mainly in skeletal muscle and adipose tissues) and an inability of pancreas to produce sufficient insulin to overcome this resistance.

Cardiovascular disease from dyslipidemia and T2D are closely associated. Insulin resistance is caused at least in part by the abnormal accumulation of lipids in the liver, muscle and other tissues. It is not yet known whether fatty liver causes insulin resistance or insulin resistance leads to storage of fat in the liver. An insulin sensitizer improves insulin's ability to stimulate cellular glucose uptake, leading to reduced levels of plasma glucose (e.g. Philip A Carpino and David Hepworth, “Beyond PPARs and Metformin: New Insulin Sensitizers for the Treatment of Type 2 Diabetes”, Annual Reports in Medicinal Chem., 47, 177-192 (2012)). Thus, it appears possible that by decreasing insulin resistance, and concomitantly, also lowering levels of circulating low density lipid particles, the dual benefits of treating T2D, and improved cardiovascular health may be realized.

The thiazolidinediones (TZDs), such as troglitazone, (RS)-5-(4-[(6-hydroxy-2,5,7,8-tetramethylchroman-2-yl)methoxy]benzyl)thiazolidine-2,4-dione, (Rezulin®, trademark of WARNER-LAMBERT COMPANY), rosiglitazone, (RS)-5-[4-(2-[methyl(pyridin-2-yl)amino]ethoxy)benzyl]thiazolidine-2,4-dione (Avandia®, trademark of SMITHKLINE BEECHAM), and pioglitazone, (RS)-5-(4-[2-(5-ethylpyridin-2-yl)ethoxy]benzyl)thiazolidine-2,4-dione (Actos®, trademark of Takeda Pharmaceutical Company) are highly effective insulin sensitizers that were approved by the FDA and market launched in the 1990s for treating T2D. The TZDs, as a class, are peroxisome proliferator-activated receptor gamma (PPARγ) agonists. However, the safety profiles of the above glitazones, after wide-spread use, led to an early withdrawal of troglitazone (idiosyncratic hepatitis); and after more than a decade of rosiglitazone use, it was withdrawn due to concerns of coronary heart disease. Pioglitazone continues to be prescribed in the US with monitoring of its potential side-effects on cardiac health and bladder cancer (Wikipedia). There is presently a need for effective and safe insulin sensitizers. Interestingly, in a clinical study of a combination therapy of fenofibrate, which lowers triglycerides and raises HDL, and rosiglitazone, paradoxically and unexpectedly, a substantial fall in HDL levels was observed (Lena Normen et al., Diabetes Care, 27(9), 2241-2242 (September 2004)).

US Patent Appln. 2006/10211749 A1 discloses a pharmaceutical composition comprising a PPARα agonist, fenofibrate, in a solvent system having omega-3 fatty acids DHA and EPA, used for treating hypertriglyceridemia, hypercholesteremia, diabetes:, and other metabolic conditions.

In 2013 the FDA approved the Takeda drug alogliptin (2-({6-[(3R)-3-aminopiperidin-1-yl]-3-methyl-2,4-dioxo-3,4-dihydropyrimidin-1(2H)-yl}methyl)benzonitrile, a dipeptidyl peptidase-4 inhibitor (DPP-4), to treat T2D in three formulations: 1) as a stand-alone with the brand-name Nesina® (trademark of Takeda Co.); 2) combined with metformin using the name Kazano® (trademark of Takeda Co.), and 3) when combined with pioglitazone using the name Oseni® (trademark of Takeda Co.). Also Takeda Co., the inventor of pioglitazone, has reintroduced the combination, Oseni® (trademark of Takeda Co.), as a safer alternative to the largely withdrawn piogltazone alone.

### Ascorbic Acid (AA)

Ascorbic acid or Vitamin C, is essential to a healthy diet, and is a highly effective antioxidant to reduce oxidative stress by acting as a reducing agent, by donating an electron to harmful, reactive, one electron free radicals thereby keeping them from entering into pathological pathways (Padayatty, Sebastian J. et al., “Vitamin C as an antioxidant evaluation of its role in disease prevention”, J. of the Am. College of Nutrition, 22(1), 18-35 (2003)). Multi-gram mega doses of vitamin C are in common use to boost the immune system, have therapeutic benefits in preventing cardiovascular disease, hypertension, chronic inflammatory diseases, and diabetes. Vitamin C's properties as an infection fighting, antioxidant-rich dietary supplement have currently turned it into the most popular topical skin care ingredient in the cosmeceutical market. WO2006019186 (Fukami, Harukazu, et al.) claims skin care products containing 6-O-dihomo-γ-linolenoyl ascorbate, 6-O-arachidonoyl ascorbate, and 6-O-docosahexaenoyl ascorbate as ingredients in an amount of 0.001-10 wt % in a base for cosmetics for external applications, which allows improved permeability of ascorbic acid to the epidermis. The same patent application also claims, 6-O-dihomo-γ-linolenoyl ascorbate, 6-O-arachidonoyl ascorbate, and 6-O-docosahexaenoyl ascorbate as ingredients in foods, sports drinks, and vitamin supplements for infants and the elderly.

Antioxidants continue to be associated with improving memory. As per clinical investigations of Pete, et al. in U.S. Pat. No. 8,188,146, omega-3 fatty acids may be tied to mental health, in addition to cardiovascular benefits.

Clearly, both omega-3 fatty acids and antioxidants, such as ascorbic acid, have beneficial properties in the treatment of various disorders; however, finding a better way to administer such ingredients is desired.

### BRIEF SUMMARY OF THE INVENTION

This invention provides a compound comprising ascorbate ester derivative compounds of the formula:

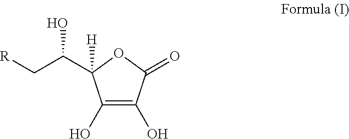

wherein: R is joined in Formula (I) from the oxygen of the carboxylate of cis, cis, cis-7,10,13-hexadecatrienoic acid (HTA), cis,cis,cis-9,12,15-octadecatrienoic acid (ALA), cis,cis,cis,cis-6,9,12,15-octadecatetraenoic acid (SDA), cis,cis,cis-11,14,17-eicosatrienoic acid (ETE), cis,cis,cis,cis-8,11,14,17-eicosatetraenoic acid (ETA), cis,cis,cis,cis,cis-5,8,11,14,17-eicosapentanenoic acid (EPA), cis,cis,cis,cis,cis-6,9,12,15,18-heneicosapentaenoic acid (HPA), cis,cis,cis,cis,cis-7,10,13,16,19-docosapentaenoic acid (DPA), cis,cis,cis,cis,cis,cis-4,7,10,13,16,19-docosahexacnoic acid (DHA), cis,cis,cis,cis,cis-9,12,15,18,21-tetracosapentaeonic acid (TPA) or cis,cis,cis,cis,cis,cis-6,9,12,15,18,21-tetracosahexaeonic acid (THA).

The pharmaceutically-acceptable formulation of the compounds of Formula (I) or its salts with pharmaceutically-acceptable adjuvants, binders, desiccants, diluents and excipients is also within this invention. This formulation containing ascorbate ester derivative compounds of Formula (I) may be administered as tablets (coated or uncoated), gummies, chocolates, ampoules, ointments, gels, suspensions, emulsions, injections (e.g., intramuscular, intravenous, intraperitoneal, subcutaneous), transdermal formulations (e.g., patches or application to the skin surface, suppository compositions), intranasal formulations (e.g., drops, sprays, inhalers, aerosol spray, chest rubs), ocular application (e.g., sterile drops, sprays, ointments), or application in a gauze, wipe, spray or other means.

Also these formulations of Formula (I) can be used in improving the effectiveness of other drugs by co-administration in treatment of diabetes, psychiatric and neurological disorders, attention deficit and hyperactivity disorder, and as cosmeceuticals.

The compounds of Formula (I) are synthesized by one of two processes:
a) In a chemical synthesis, an omega-3 fatty acid methyl or ethyl ester is hydrolyzed with one equivalent of sodium or potassium hydroxide in either methanol or ethanol. The product, as its sodium or potassium salt, is concentrated free of methanol or ethanol using toluene as an azeotropic solvent. The residue is treated with an equivalent of oxalyl chloride in refluxing dichloromethane. The resulting acid chloride of the omega-3 acid is dissolved in methylene chloride and reacted with one equivalent each of ascorbic acid and N-methylpyrollidone (NMP). After the reaction is complete, the mixture is extracted with 4N HCl to remove byproducts and, and then concentrated to dryness. The resulting omega-3 ascorbate is purified either by chromatography or recrystallization from a suitable solvent, such as acetonitrile, diisopropyl ether, toluene or other solvent; or
b) In an enzymatic synthesis, an omega-3 fatty acid methyl or ethyl ester is transesterified to the corresponding ascorbate using an immobilized lipase enzyme such as Lipase PS “Amano” IM. This is best carried out to high conversion in organic solvents such as isopropyl ether, toluene, acetone, or other solvent. The catalyst is filtered off, the filtrate concentrated and the residue purified either by chromatography or recrystallization from a suitable solvent, such as acetonitrile, diisopropyl ether, toluene or other solvent.

The compounds of Formula (I) and their formulations provide both omega-3 fatty acids and antioxidants, such as ascorbic acid, having beneficial properties in the treatment of various disorders; and a better way to administer them.

### BRIEF DESCRIPTION OF THE DRAWING

FIG. 1 is a graph of the data obtained from biology tests described herein of EPA and DHA ascorbates on fatty acid oxidation rates in cultured on normal primary human myocytes. The vertical axis is the complete oxidation (nmol CO₂/mg protein). The statistical variance is shown by the “T” at the top of each bar. The #1 bar (solid coral) is the ethanol as control; #2 bars (solid gray, from left to right are 30, 100 and 300 micromolar) of EPA; #3 bars (solid green, from left to right are 30, 100 and 300 micromolar) of EPAX; #4 bars (hash red and white, from left to right are 30, 100 and 300 micromolar) of DHAX; and #5 bars (solid red, from left to right are 30, 100 and 300 micromolar) of EPAEE.

### DETAILED DESCRIPTION OF THE INVENTION

It is understood that the terminology used herein is for the purpose of describing particular embodiments only and is not intended to be limiting. As used in this specification, the singular forms “a”, “an”, and “the” include plural referents unless the content clearly indicates otherwise. The following terms in the Glossary as used in this application are to be defined as stated below and for these terms, the singular includes the plural.

Various headings are present to aid the reader, but are not the exclusive location of all aspects of that referenced subject matter and are not to be construed as limiting the location of such discussion.

Also, certain US patents and PCT published applications have been incorporated by reference. However, the text of such patents is only incorporated by reference to the extent that no conflict exists between such text and other statements set forth herein. In the event of such conflict, then any such conflicting text in such incorporated by reference US patent or PCT application is specifically not so incorporated in this patent.

### Glossary

The fatty acid structures below are illustrating the R term of Formula (I) and the oxygen through which it joins onto Formula (I).
ALA means α-linolenic acid or cis,cis,cis-9,12,15-octadecatrienoic acid, having 18 carbons, 3 cis double bonds, of the formula

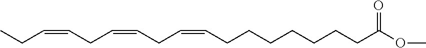 [SMILES: CC/C=C\C/C=C\C/C=C\CCCCCCCC(=O)OC]

DHA means cis,cis,cis,cis,cis,cis-4,7,10,13,16,19-docosahexaenoic acid or docosahexaenoic acid, having 22 carbons, 6 cis double bonds, of the formula

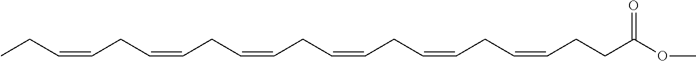 [SMILES: CC/C=C\C/C=C\C/C=C\C/C=C\C/C=C\C/C=C\CCC(=O)OC]

DPA means cis,cis,cis,cis,cis-7,10,13,16,19-docosapentaenoic acid, having 22 carbons, 5 cis double bonds, of the formula

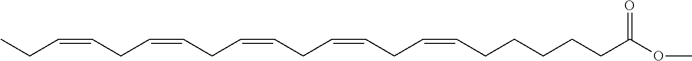 [SMILES: CC/C=C\C/C=C\C/C=C\C/C=C\C/C=C\CCCCCC(=O)OC]

EPA means cis,cis,cis,cis,cis-5,8,11,14,17-eicosapentanenoic acid or eicosapentanenoic acid, having 20 carbons, 5 cis double bonds, of the formula

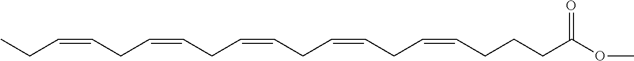 [SMILES: CC/C=C\C/C=C\C/C=C\C/C=C\C/C=C\CCCC(=O)OC]

ETA means cis,cis,cis,cis-8,11,14,17-eicosatetranoic acid or eicosatetraenoic acid, having 20 carbons, 4 cis double bonds, of the formula

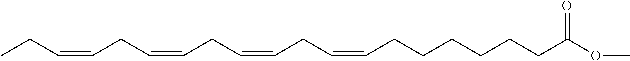 [SMILES: CC/C=C\C/C=C\C/C=C\C/C=C\CCCCCCC(=O)OC]

ETE means cis,cis,cis-11,14,17-eicosatrienoic acid or eicosatrienoic acid, having 20 carbons, 3 cis double bonds, of the formula

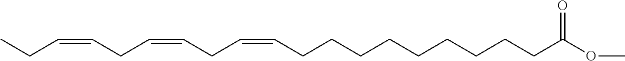 [SMILES: CC/C=C\C/C=C\C/C=C\CCCCCCCCCC(=O)OC]

HPA means cis,cis,cis,cis,cis-6,9,12,15,18-heneicosapentaenoic acid or heneicosapentaenoic acid, having 21 carbons, 5 cis double bonds, of the formula

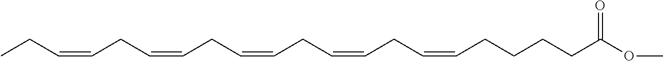 [SMILES: CC/C=C\C/C=C\C/C=C\C/C=C\C/C=C\CCCCC(=O)OC]

HTA means cis, cis, cis-7,10,13-hexadecatrienoic acid, having 16 carbons, 3 cis double bonds, of the formula

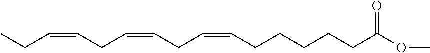 [SMILES: CC/C=C\C/C=C\C/C=C\CCCCCC(=O)OC]

SDA means cis,cis,cis,cis-6,9,12,15-octadecatetraenoic acid or stearidonic acid, having 18 carbons, 4 cis double bonds, of the formula

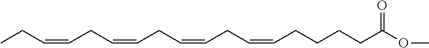 [SMILES: CC/C=C\C/C=C\C/C=C\C/C=C\CCCCC(=O)OC]

THA means cis,cis,cis,cis,cis,cis-6,9,12,15,18,21-tetracosahexaconic acid, having 24 carbons, 6 cis double bonds, of the formula

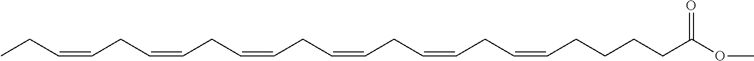 [SMILES: CC/C=C\C/C=C\C/C=C\C/C=C\C/C=C\C/C=C\CCCCC(=O)OC]

TPA means cis,cis,cis,cis,cis-9,12,15,18,21-tetracosapentaeonic acid, having 24 carbons, 5 cis double bonds, of the formula

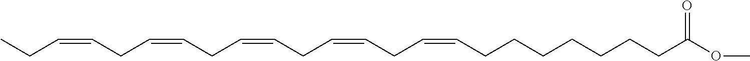 [SMILES: CC/C=C\C/C=C\C/C=C\C/C=C\C/C=C\CCCCCCCC(=O)OC]

Omega-3 fatty acids means naturally occurring, straight-chain C₁₆-C₂₄ fatty carboxylic acids
PUFA means polyunsaturated fatty acids that are either naturally occurring omega-3 fatty acids or derivatives thereof.
“AA” means ascorbic acid
“DHAX” means DHA ascorbate; a compound of Formula (I)
“EPA” means EPA fatty acid; the structure above for EPA but with an OH at the terminal shown for attachment
“EPAEE” means EPA ethyl ester; the structure above for EPA but with an ethyl group at the terminal shown for attachment
“EPAX” means EPA ascorbate; a compound of Formula (I)
“g” means grain
“hr” means hour
“min” means minute
“mL” means milliter
“overnight” means about 18 to about 24 hours
“RT” means room temperature, about 22 to about 26° C.
“TFA” means trifluoroacetic acid
“THF” means tetrahydrofuran

### General Discussion

This invention discusses the potential therapeutic benefits of agents made by conjugating omega-3 fatty acids with antioxidants, such as Vitamin C and Vitamin E, either singly, or in combination with other therapies to offer greater long-term cardiovascular benefits. In particular, the conjugates made from PUFAs and Vitamin C (ascorbic acid) are of special interest, as they are low melting solids, have significant aqueous solubility, and unique pharmacokinetic properties, compared to their water insoluble PUFA ethyl esters, which are currently in use to treat hypertriglycerdemia. These formulations of ascorbate ester derivative compounds of Formula (I) have better taste, minimal fishy odors, and are suitable for alternative formulations such as tablets, capsules, gummies and other dosage forms. These formulations are under further evaluation for these purposes.

While not wishing to be bound by theory, it is believed that the compounds of Formula (I) can provide all these benefits for the following reasons. Ascorbic acid, derived from glucose, is a weak acid with a pKa=4.1, with an aqueous solubility of 2.57 g/mL. Its acidity stems from the bis-enol-γ-lactone structure, with the enol providing the ionizable proton. The side-chain has both a primary and a secondary alcohol functionality that are suitable for conjugating with the omega-3 fatty acids. The primary alcohol group in the 6-position is more accessible and the more reactive site for synthesis of compounds of Formula (I).

Also 6-O-palmitoyl L-ascorbate (with 16 carbons, no unsaturation), MW 414.533, is commercially available (Sigma-Aldrich). Palmitoyl ascorbate is a solid, mp 113-116° C., sparingly soluble in water, but soluble in aqueous ethanol. The molecular weights of ALA-ascorbate, DHA-ascorbate, and EPA ascorbate are 436.571; 486.608, and 460.608, respectively. These molecular weights are in the same range as the palmitoyl ascorbate, and are low melting solids when pure.

The incorporation of a conjugated antioxidant pharmacophore is previously unknown, and would lead to improved therapeutic benefits, and insights into the mechanisms of action of omega-3 fatty acids in treating, neurological disorders in children such as ADHD; hypertriglycerdemia and diabetes in adults and juvenile diabetes. The relatively large doses of omega-3 fatty acids and ascorbic acid individually in current use are reasonably well mass-balanced in the conjugates of ascorbate ester derivative compounds of Formula (I), e.g., in the EPA ascorbate compound of Formula (I) has a molecular weight of 460.608; EPA accounts for 302.451, and ascorbic acid, 176.12, minus 18.014, a mole of water. For example, it is plausible that these medications, in combination with an appropriate omega-3 supplement, such as a DHA derivative, would provide children with ADHD enhanced learning and social behavioral outcomes. This is another purpose of the invention. Since the doses of various ADHD drugs are usually small, 5-50 mg/day, and the dose of an omega-3 supplement is probably ≧100 mg/day, using alternative formulations, such as a combination gummy, or a liquid-gel capsule may be appropriate. Additionally, it seems plausible, that in combination with a lipid lowering agent, rosiglitazone and pioglitazone, used at clinical doses lower than those that have been widely prescribed previously, may safely be returned to market without displaying their cardiovascular and other risks.

Formula (I) PUFA ascorbates' applications of use and reasons for such use are discussed below:

a) As Nutritional Dietary Supplements:
Nordic Naturals markets a baby omega-3 supplement (Baby's DHA) that claims to help support learning and language abilities and healthier brain and eyes development. Clinical studies in 4 year old children support the beneficial effects of docohexaenoic acid (DHA) on cognitive function (NCT 00351624; 2006-2008; sponsored by Martek BioSciences Corporation). The Food and Nutrition Boards in the United States and in Canada have established recommended Adequate Intakes (AI values) of omega-3 fatty acids ALA, DHA and EPA for infants, children, pregnant women and adults. These values are publically available. The ALA, DHA, and EPA ascorbates, with their aqueous solubility and absence from fishy odors, would offer an opportunity to create novel fruit flavored formulations that babies would enjoy the taste, as liquid drops, and children and adults in the form of gummies Thus the formulation of Formula (I) ascorbates of ALA, DHA, and EPA ascorbates are intended as neutraceuticals in the manner of fish oils.

b) Treating Hypertriglycerdemia:
Pharmaceutically effective mixtures of ethyl esters of eicosapentaenoic acid and docosahexaneoic acid are clinically prescribed to treat hypertriglyceridemia. For example, the drug Lovaza™, developed by Reliant Pharmaceuticals and marketed by GlaxoSmithKline (GSK), is approved by the US FDA to lower very high triglyceride levels ≧500 mg/dl. In July 2012, the US FDA approved Amarin's Vascepa™ (icosapent ethyl, EPA ethyl ester) for treating severe hypertriglyceridemia (U.S. Pat. No. 8,188,146). As an example from the present invention, the water soluble EPA ascorbate is believed to be a novel therapeutic molecular entity to treat severe hypertriglycerdemia of ≧500 mg/dl in humans. Based on the therapeutic properties of the compounds of Formula (I), these compounds would demonstrate enhanced long-term cardioprotective benefits from the conjugated ascorbic acid pharmacophore, and certainly is believed to offer alternative, more desirable delivery formulations due to its aqueous solubility. Thus the formulation of EPA ascorbate of Formula (I) is a preferred formulation for this utility.

c) Type2 Diabetes:
Worldwide, there is a very rapid rise in the incidence of T2D, even in preteen children, due to high carbohydrate diet, and obesity. This is a major health concern in the next decades (Wall Street Journal, Jan. 28, 2013, “Grim New Diabetes Milestone”).
The most anticipated application of PUFA ascorbates of Formula (I) would be as combination therapies with metformin, and/or thioglitazones such as rosiglitazone and pioglitazone. As noted above on page 5, line 8-11, in a clinical study of combination therapy of fenofibrate, which lowers triglycerides and raises HDL, and rosiglitazone, paradoxically and unexpectedly resulted in a substantial fall in HDL levels (Lena Normen et al., Diabetes Care, 27(9), 2241-2242 (September 2004)). However, unlike fenofibrate, which is a peroxisome proliferator-activator receptor alpha (PPARα) agonist, rosiglitazone is a PPARγ activator. (Both receptors have a distinct tissue expression. PPARα is expressed at high levels in the liver, whereas PPARγ is expressed in many tissues, with the highest concentrations in adipose and skeletal muscle cells).
Both of the omega-3 acids, DHA and EPA, are PPARγ agonists. None of the authors of the prior art discussed above have investigated combining two structurally distinct PPARγ agonists, such as an omega-3 acid or a beneficial derivative thereof, like the present ascorbate of Formula (I), with a clinically useful thiazolidinedione, such as rosiglitazone or pioglitazone, in treating T2D, and possibly also assuring safe cardiovascular health and minimizing other side-effects. Thus a formulation of one tablet or other suitable dosage form having both an ascorbate of Formula (I) and metformin and/or a thiazolidinedione such as rosiglitazone or pioglitazone is believed valuable.

d) Attention Deficit Hyperactivity Disorder Treatment:
Currently, attention is drawn to the possible benefits of omega-3 supplements in treating school children of 6-12 years age, as well as teenagers, who are diagnosed with symptoms of attention deficit hyperactivity disorder (ADHD). Prescribed treatments for ADHD are various stimulant medications, such as atomoxetine hydrochloride (Concerta®, trademark of ALZA Corporation), methylphenidate (Ritalin®, trademark of NOVARTIS CORPORATION), dextroamphetamine plus amphetamines (Adderall®, trademark of SHIRE LLC), and lisdexamfetamine (Vynanse®, trademark of SHIRE LLC), and others. It is plausible that these medications, in combination with an appropriate omega-3 supplement, such as a DHA ascorbate of Formula (I), would provide children with ADHD enhanced learning and social behavioral outcomes. Since the doses of various ADHD drugs are usually small, 5-50 mg/day, and the dose of an omega-3 supplement is probably ≧100 mg/day, a formulation having both the ascorbate of Formula (I) and the ADHD drug present, such as a gummy, may be appropriate.

While not wishing to be bound by the above theory, it is believed that the formulations of the ascorbate ester derivative compounds of Formula (I) may be used in a similar manner for omega-3 fatty acids such (a) as an essential development nutrient from infants to teen agers, and pregnant women of all ages, (b) in treating hypertriglyceridemia, (c) diabetes, Type2 diabetes in combination with PPARγ agents such as rosoglitazone or pioglitazone in order to improve their overall cardiovascular safety profiles, (d) psychiatric and neurological disorders, in treating ADHD, and (e) as cosmeceuticals. For example the present formulation having an ascorbate ester derivative compound of Formula (I) can be included in a multi-drug treatment with any drug in use for the treatment of psychiatric and neurological disorders to improve the efficacy of the drug. These drugs are for treating depression (cetalopram), schizophrenia (risperidone), Alzheimer's (memantine), and ADHD (Ritalin®, Adderall®, and Concerta®) and others now in clinical use.

This invention provides a compound comprising ascorbate ester derivative compounds of the formula:

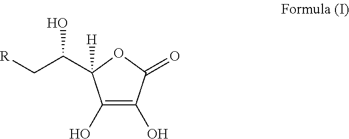

wherein: R is joined in Formula (I) from the oxygen of the carboxylate of cis, cis, cis-7,10,13-hexadecatrienoic acid (HTA), cis,cis,cis-9,12,15-octadecatrienoic acid (ALA), cis,cis,cis,cis-6,9,12,15-octadecatetraenoic acid (SDA), cis,cis,cis-11,14,17-eicosatrienoic acid (ETE), cis,cis,cis,cis-8,11,14,17-eicosatetraenoic acid (ETA), cis,cis,cis,cis,cis-5,8,11,14,17-eicosapentanenoic acid (EPA), cis,cis,cis,cis,cis-6,9,12,15,18-heneicosapentaenoic acid (HPA), cis,cis,cis,cis,cis-7,10,13,16,19-docosapentaenoic acid (DPA), cis,cis,cis,cis,cis,cis-4,7,10,13,16,19-docosahexaenoic acid (DHA), cis,cis,cis,cis,cis-9,12,15,18,21-tetracosapentaeonic acid (TPA) or cis,cis,cis,cis,cis,cis-6,9,12,15,18,21-tetracosahexaeonic acid (THA).

The omega-3 fatty acid is joined to the ascorbate compound through its distal carboxylic acid group forming the ester. While not wishing to be bound by theory, it is believed that the ascorbate portion of the compound of Formula (I) provides a greater aqueous solubility for the omega-fatty acid, while the fatty acid chain provides a lipid character to the compound of Formula (I). These features enable the ascorbate ester derivative compound of Formula (I) to provide better bioavailability for drug absorption; and absorption and penetration through the epidermis by the compound. It combines the features of both an antioxidant and the omega-3 fatty acid thereby providing for unique and multiple mechanisms of action.

The omega-3 ascorbate solids allow them to be purified to a very high degree >95% by conventional crystallization/purification techniques or by sophisticated preparative/production hplc methods, such as simulated moving bed chromatography.

The polyunsaturated fatty acids (PUFA) ascorbate esters represented by Formula (I) may be prepared by a number of known methods such as:
1) Using enzymatic immobilized lipase chirazyme L-2 catalysis from the methods described by Watanabe, Yoshiyuki, et al., J. Am. Oil Chemists' Society, 78(8), 823-826 (2001) or by the method in Biotechnology Letters 22(8), 637-640 (2000); or
2) By chemical synthesis via reactive PUFA acid chloride intermediates as described by Yazawa, Kazuyoshi, et al., Japan Kokai Tokkyo Koho (1994). Additionally, Fukami, Harukazu; Kawashima, Hiroshi; Skin Care Products, As Well As Foods And Beverages Containing 6-O-PUFA Ascorbic Esters in WO2006/019168. WO 2006019186 describes the preparation of 6-O-arachidonoyl ascorbate and 6-O-docosohexaneoyl ascorbate as pastes, however no purity was indicated.

This invention will be further clarified by a consideration of the following examples, which are intended to be purely exemplary of the present invention. The examples for EPA-L-ascorbate and DHA-L-ascorbate are generally applicable to all PUFAs.

### EXAMPLE 1

The eicocpentaenoic (EPA) and docosohexaenoic (DHA) esters of L-ascorbic acid were prepared by the using the methodology found in the above patent literature for the arachidonic ester of ascorbic acid. To reduce the cost and verify the chemistry would work for EPA, low purity EPA ethyl ester (65%) was hydrolyzed to the corresponding sodium salt with sodium hydroxide. The salt was then treated with oxalyl chloride to form the acid chloride. The acid chloride was coupled with L-ascorbic acid to prepare the EPA-ascorbate ester. The synthesis of the EPA ascorbate ester is outlined in Scheme 1.

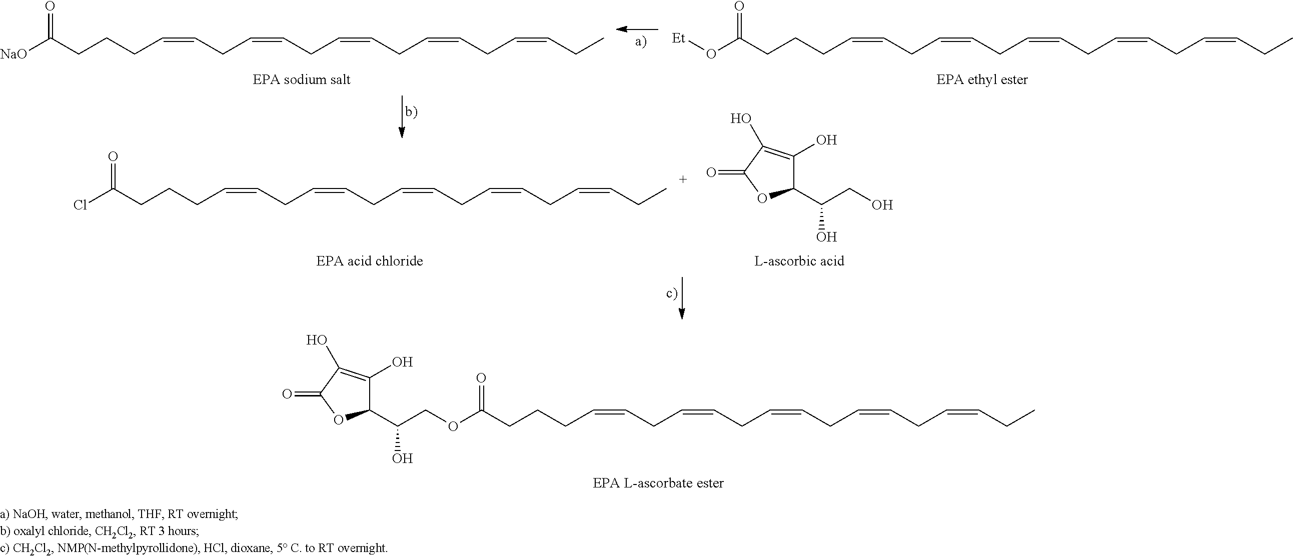

### Detailed Procedure of Scheme 1.

EPA ethyl ester (65%, TCI America, 10.0 g, 0.03 mol) was mixed with THF (40 mL), methanol (40 mL), and water (50 mL) containing sodium hydroxide (1.21 g, 0.03 mol) at RT, overnight under an argon atmosphere. After 20 hr, the solution was concentrated on a rotary evaporator. Methanol (100 mL) was added and the mixture concentrated. Fresh methanol (100 mL) was added and the mixture concentrated. The process was repeated with dichloromethane (100 mL) and toluene (100 mL). The remaining light brown solid was dried under high vacuum at RT overnight. The dried solid was mixed with anhydrous dichloromethane (100 mL) and oxalyl chloride (6.0 g, 0.047 mol). After 3 hr the mixture was filtered and concentrated to prepare the acid chloride of EPA (9.4 g, 0.029 mol) as a tan oil. In a separate flask, L-ascorbic acid (6.0 g, 0.034 mol) was added to N-methylpyrrolidone (65 mL) that contained hydrogen chloride in dioxane (1.1 mL of 4N HCl in dioxane, Aldrich) at 5° C. (ice/water bath), under an argon atmosphere. After 20 min at 5° C., the acid chloride of EPA (9.4 g, 0.029 mol) in dichloromethane (5 mL) was added drop-wise over 5 min. The solution was allowed to stir for 4 hr at 5° C., then slowly warmed to RT and stirred at RT overnight. After 22 hr, the solution was added to water (200 mL) and the product was extracted with ethyl acetate (200 mL). The ethyl acetate solution was washed with water (2×150 mL), dried over sodium sulfate, filtered and concentrated. The crude product (13.3 g, brown oil) was purified on silica gel (320 g) eluting with ethyl acetate/heptanes (3:2) to generate the EPA-AA ester (5.74 g, 0.0125 mol) as a tan gel/glass that was 70% pure by HPLC.

To further purify the EPA-AA ester, portions (1.8 g) were purified twice by reverse phase chromatography on a C18 cartridge (100 g) using an automated MPLC system (Combi-flash), eluting with 40-90% methanol/water over 24 min and 90% methanol/water over 20 min (observing at 233 nm, RT=26-28 min.). The MPLC purification, after concentration and drying, generated 0.7 g light tan gel that was pure EPA-AA ester (98% purity, HPLC). The EPA-AA has the following characteristics:

Appearance: light tan gel/glass

Chemical Formula: C₂₆H₃₆O₇

Molecular Weight: 460.56

Chromatographic purity (HPLC): 98.0% (RT=10.867 min, 72-95% MeOH/H₂O (TFA 0.1%) over 10 min, Luna C18, 5, 4.6×250 mm, 1.0 mL/min, 10 L injection, 40° C., UV detection, 230 nm)

HRMS (MMI-TOF-MS): Calculated for C₂₅H₃₆O₇ (M−H)⁻: 459.2388; found: 459.2399

¹H NMR (300 MHz, CDCl₃/TMS): δ 5.44-5.27 (m, 10H), 4.80 (s, 1H, br), 4.45-4.25 (m, 3H), 2.90-2.75 (m, 8H), 2.38 (t, 2H, J=7.5 Hz), 2.15-2.00 (m, 4H), 1.71 (m, 2H), 0.97 (t, 3H J=7.2 Hz). Conforms to expected structure.

¹³C NMR (75 MHz, CDCl₃/TMS): δ 174.0, 172.2, 152.4, 132.2, 129.2, 128.9, 128.8, 128.5, 128.4, 128.3, 128.2, 128.0, 127.2, 119.1, 76.3, 68.1, 64.5, 33.9, 26.9, 26.0, 25.9, 25.0, 20.9, 14.6. Conforms to expected structure.

### EXAMPLE 2

### Synthesis of DHA-AA (Structure Below) is as Follows.

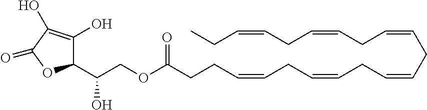 [SMILES: CC/C=C\C/C=C\C/C=C\C/C=C\C/C=C\C/C=C\CCC(=O)OC[C@H](O)[C@H]1OC(=O)C(O)=C1O]

DHA acid (1.0 g, 3.0 mmol, 98% purity, Acros Organics) was dissolved in dichloromethane (10 mL) under an argon atmosphere, at RT. To the DHA solution was added oxalyl chloride (0.7 g, 5.5 mmol). After stirring for 3 hr at RT, the solution was concentrated under reduced pressure to prepare the acid chloride of DHA. The acid chloride of DHA was dissolved in dichloromethane (4 mL) and added to a mixture of N-methylpyrrolidone (10 mL), hydrogen chloride in dioxane (1 mL of 4N), and L-ascorbic acid (0.8 g, 4.5 mmol) stirred at 5° C. under an argon atmosphere. The solution stirred for 5 hr at 5° C., slowly warmed to RT, and stirred at RT overnight. After 22 hr, ethyl acetate (100 mL) was added and the product was washed with water (3×50 mL), dried over sodium sulfate, filtered and concentrated. The crude product (1.4 g tan gel) was purified on silica gel (25 g) eluting with ethyl acetate/heptanes (70:30) to generate the EPA-AA ester (0.6 g, 1.23 mmol) as a tan gel/glass that was 96.1% pure by HPLC. The EPA-AA has the following characteristics:

### Analysis:

Appearance: light tan gel/glass

Chemical Formula: C₂₈H₃₈O₇

Molecular Weight: 486.60

Chromatographic purity (HPLC): 96.1% (RT=14.315 min, 80-100% MeOH/H₂O (TFA 0.1%) over 10 minutes, Luna C18, 5, 4.6×250 mm, 1.0 mL/min, 10 L injection, 25° C., UV detection, 230 nm)

HRMS (MMI-TOF-MS): Calculated for C₂₈H₃₇O₇ (M−H)⁻: 485.2545; found: 485.2548

¹H NMR (300 MHz, CDCl₃/TMS): δ 5.45-5.26 (m, 12H), 4.80 (s, 1H, br), 4.44 (m, 1H), 4.27 (m, 2H), 2.90-2.75 (m, 10H), 2.50-2.35 (m, 4H), 2.07 (m, 2H), 0.97 (t, 3H J=7.5 Hz).

¹³C NMR (75 MHz, CDCl₃/TMS): δ 173.5, 171.9, 151.7, 132.2, 129.8, 128.8, 128.6, 128.5, 128.3, 128.2, 128.1, 127.8, 127.2, 119.3, 76.2, 68.4, 64.5, 34.3, 26.0, 23.0, 20.9, 14.6.

### Biology of Omega-3 Ascorbates:

Effect of EPA and DHA ascorbates on fatty acid oxidation rates in cultured primary human myocytes were determined and discussed below.

It has been shown that primary skeletal muscle cells from patients with type 2 diabetes as well as severe obesity have reduced capacity to oxidize fatty acids compared with cells isolated from healthy controls (e.g., Gaster, M., et al., Reduced lipid oxidation in skeletal muscle from type 2 diabetic subjects may be of genetic origin: evidence from cultured myotubes. Diabetes, 53, 542-548 (2004); Cha, B. S., et al., Impaired fatty acid metabolism in type 2 diabetic skeletal muscle cells is reversed by PPARgamma agonists, Am. J. Physiol. Endocrinol. Metab., 289, E151E159 (2005); and Hulver, M. W., et al. 2005. Elevated stearoyl-CoA desaturase-1 expression in skeletal muscle contributes to abnormal fatty acid partitioning in obese humans, Cell Metab. 2, 251-261 (2005)). Moreover, there are clear correlations between metabolic parameters related to diabetes, such as insulin sensitivity, body fat mass, and plasma lipid levels, with fatty acid oxidation measured in primary cells from respective donors (Ukropcova, B., M. et al., Dynamic changes in fat oxidation in human primary myocytes mirror metabolic characteristics of the donor, J. Clin. Invest. 115, 1934-1941(2005)). These and many similar observations strongly suggest that the effect of a pharmacological agent on fatty acid oxidation in cultured primary skeletal muscle cells would be predictive of its metabolic effects in patients. In particular, a compound that stimulates fatty acid oxidation in this in vitro system would be predicted to reduce plasma lipids and improve metabolic health in patients.

Cell-based multiwell assays for the detection of substrate accumulation and oxidation are described in literature (A. J. Wensaas, et al., J. of Lipid Research 48, 961-967 (2007)) for detecting the accumulation as well as the subsequent oxidation of ¹⁴C labeled substrates in cultured cells. Accumulation is monitored in real time by an established scintillation proximity assay in which the scintillator is embedded in the plate base primarily detecting cell-associated radiolabel. This method was applied, and the effect of EPA and DHA ascorbates on fatty acid oxidation rates in cultured primary human myocytes was determined The compounds tested were EPA, EPAX (EPAX), DHAX (DHAX), and EPAEE.

### Summary of Assay Parameters;

Cultured human primary myocytes from normal (non-diabetic, non-obese) subjects

Cells treated with 30, 100 and 300 micromolar of each compound for 48 hr

Cells grown in the presence of radiolabeled oleic acid

Radiolabeled CO₂ released during the incubation was measured.

Each compound was tested 6 times (n=6)

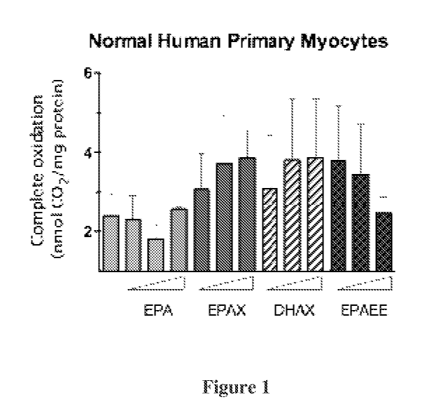

The data is summarized in FIG. 1. The following conclusions from these results are made.

There is a distinct trend for EPA ascorbate, and to a lesser degree DHA ascorbate, to promote fatty acid oxidation, especially as compared to EPAEE (ethyl ester). However, differences from control are not statistically significant.

It should be recognized that the ascorbate esters of omega-3 fatty acids are prodrugs, just as their ethyl esters are. These ascorbate esters are not expected to be better in their potential to stimulate fatty acid oxidation from their ethyl esters; but they might be somewhat better from this data. However, the other anticipated cardioprotective benefits of omega-3 ascorbates would still need to be determined from other clinical studies.

### Formulations

A pharmaceutically-acceptable formulation of the compounds of Formula (I) or its salts is included in this invention. These ascorbate ester derivative compounds of Formula (I) are formulated into various pharmaceutically-acceptable forms. A tablet may be made using binders known to those skilled in the art. Such dosage forms are described in Remington's Pharmaceutical Sciences, 18^{th} ed. 1990, pub. Mack Publishing Company, Easton, Pa. Suitable tablets include compressed tablets, sugar-coated tablets, film-coated tablets, enteric-coated tablets, multiple compressed tablets, gummies, chocolates, controlled-release tablets, and the like. Ampoules, ointments, gels, capsules, suspensions, emulsions, injections (e.g., intramuscular, intravenous, intraperitoneal, subcutaneous), transdermal formulation (e.g., patches or application to the skin surface, suppository compositions), intranasal formulations (e.g., drops, sprays, inhalers, aerosol spray, chest rubs), and ocular application (e.g., sterile drops, sprays, ointments) may also be used as a suitable formulation.

Customary pharmaceutically-acceptable salts, adjuvants, binders, desiccants, diluents and excipients are used in these formulations.

Another advantage of the omega-3 ascorbates of Formula (I) is that the aqueous solutions may be administered as IV solutions to treat acute cases of hypertriglyceridemia.

The formulations of the ascorbate ester derivative compounds of Formula (I) may be used as an essential development nutrient from infants to teen agers, and pregnant women of all ages, in treating hypertriglyceridemia, diabetes, Type2 diabetes in combination with PPARγ agents such as rosoglitazone, pioglilazone in order to improve their overall cardiovascular safety profiles, psychiatric and neurological disorders, in treating ADHD, and as cosmeceuticals. For example the present formulation having an ascorbate ester derivative compound of Formula (I) can be included in a multi-drug treatment with any drug in use for the treatment of psychiatric and neurological disorders to improve the efficacy of the drug. These drugs are for treating depression (cetalopram), schizophrenia (risperidone), Alzheimer's (memantine), and ADHD (Ritalin®, Adderall®, and Concerta®) and others in clinical use.

The formulation of the ascorbate ester derivative compounds of Formula (I) is of ≧50% chemical purity, preferably ≧90% chemical purity, more preferably ≧95% chemical purity.

Preferably the formulation of ascorbate ester derivative compounds of Formula (I) are those where R is ALA, DHA, DPA or EPA, each of which is ≧95% chemical purity. These formulations of Formula (I) can be used as:

1) a nutritional and dietary supplement for an adult, wherein the dose is from about 0.5 to about 5 g/day in 1-4 doses/day;

2) an essential developmental nutritional supplement for newborn infants, children up to 18 years, and pregnant women of all ages, wherein the dose is from about 0.05 g to about 1.6 g/day; especially preferred are the formulations wherein the R of Formula (I) is DHA, and the dose is from about 50 mg to 500 mg/day, and wherein two or more compounds of Formula (I) are present in a single formulation, especially those where the R of Formula (I) is DHA, DPA and EPA, and the combined dose of the three compounds is from about 50 mg to 500 mg/day;

3) reduction of triglycerides in a human in need thereof, having fasting triglyceride level from ≧200 mg/dl to ≧500 mg/dl and up to1500 mg/dl; preferably using compounds of Formula (I) wherein R is EPA and the dose is from about 0.5 to about 5 g/day in 1-4 doses/day;

4) as a therapeutic treatment of Type 2 diabetes, defined by fasting plasma glucose of ≧7.0 mmol/l (126 mg/dl), and/or glycated hemoglobin (HbA_{1c}) of greater than 6.5%; or used in a combination therapy as single formulation of the ascorbate of Formula (I) with metformin, about 500 mg, as 2 doses/day;

5) a component in a combination of a single formulation therapy with the ascorbate of Formula (I) and a dose of ≦2-8 mg of rosiglitazone maleate or with a dose of ≦15-30 mg of pioglitazone hydrochloride for the therapeutic treatment of Type 2 diabetes with concomitant reduction in hypertriglyecridemia, and improved long-term cardiac and other health outcomes;

6) a component in a dose of 50-1000 mg/day in a combination of a single formulation therapy of an ascorbate of Formula (I) with commonly prescribed doses of medications such as atomoxetine hydrochloride (Concerta®), methylphenidate (Ritalin®), dextroamphetamine plus amphetamines (Adderall®) or lisdexamfetamine (Vynanse®) to treat attention deficit hyperactivity disorder in children 6-12 years and teenagers;

7) as a component of a therapy with known treatments for psychiatric and neurological disorders such as depression, schizophrenia, bipolar disorder, attention deficit hyperactivity disorder, Alzheimer's disease; and

8) as one of the components in a topical formulation used as sunscreen lotions, facial and skin care lotions, cleansers, ointments, cosmetics, or scalp and hair care products, including hair growth.

Additionally, mixtures of 2 or more ascorbate ester derivative compounds of Formula (I) can be used in the above formulation. Preferred is a mixture of two ascorbates of Formula (I) wherein the R of Formula (I) is DHA and EPA and the ratio of DHA to EPA ascorbates in the formulation by weight is from about 93:7 to about 3:97. If desired, the formulation of Formula (I) can be administered concurrently with the other active drug mentioned above, but it is preferably in a single formulation.

Although the invention has been described with reference to its preferred embodiments, those of ordinary skill in the art may, upon reading and understanding this disclosure, appreciate changes and modifications which may be made which do not depart from the scope and spirit of the invention as described above or claimed hereafter.

1. A compound comprising an ascorbate ester derivative compound of the formula 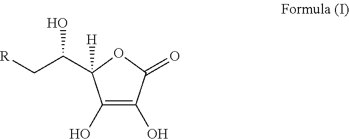
wherein; R is joined in Formula (I) from the oxygen of the carboxylate of cis, cis, cis-7,10,13-hexadecatrienoic acid (HTA), cis,cis,cis-9,12,15-octadecatrienoic acid (ALA), cis,cis,cis,cis-6,9,12,15-octadecatetraenoic acid (SDA), cis,cis,cis-11,14,17-eicosatrienoic acid (ETE), cis,cis,cis,cis-8,11,14,17-eicosatetraenoic acid (ETA), cis,cis,cis,cis,cis-5,8,11,14,17-eicosapentanenoic acid (EPA), cis,cis,cis,cis,cis-6,9,12,15,18-heneicosapentaenoic acid (HPA), cis,cis,cis,cis,cis-7,10,13,16,19-decosapentaenoic acid (DPA), cis,cis,cis,cis,cis,cis-4,7,10,13,16,19-docosahexaenoic acid (DHA), cis,cis,cis,cis,cis-9,12,15,18,21-tetracosapentaeonic acid (TPA) or cis,cis,cis,cis,cis,cis-6,9,12,15 8,21-tetracosahexaeonic acid (THA).

2. The compound of claim 1, wherein each ascorbate ester derivative compound of Formula (I) is ≧90% chemically pure.

3. The compound of claim 1 wherein the R of Formula (I) is ALA, DHA, DPA or EPA, each of which is ≧90% chemically pure.

4. A pharmaceutically-acceptable formulation comprising an ascorbate ester derivative compound of Formula (I) as defined in claim 1, or its pharmaceutically-acceptable salts, with pharmaceutically-acceptable adjuvants, binders, desiccants, diluents and excipients.

5. The formulation of claim 4 wherein the R of Formula (I) is ALA, DHA, DPA or EPA, each of which is ≧90% chemically pure.

6. The formulation of claim 4 in the form of a solution for injection, soft or hard gelatin capsules, liquid gel caplets, tablets or gummies.

7. The formulation of claim 4 wherein each ascorbate ester derivative compound of Formula (I) is ≧90% chemically pure.

8. (canceled)

9. The formulation of claim 4 wherein the R of Formula (I) is ALA, DHA, DPA or EPA, each of which is ≧90% chemically pure.

10. The formulation of claim 9 wherein the formulation contains two ascorbates of Formula (I) wherein the R of Formula (I) is DHA and EPA and the ratio of DHA to EPA ascorbates in the formulation by weight is from 97:3 to 3:97.

11. The formulation of claim 9 as a nutritional and dietary supplement for an adult, wherein the dose is from about 0.25 to about 5 g/day in 1-4 doses/day.

12. The formulation of claim 9 as an essential developmental nutritional supplement for newborn infants, children up to 18 years, and pregnant women of all ages, wherein the dose is from about 0.05 g to about 1.6 g/day.

13. The formulation of claim 12, wherein the R of Formula (I) is DHA, and the dose is from about 50 mg to 500 mg/day.

14. (canceled)

15. The formulation of claim 10, wherein the formulation is used in concert with prescribed treatments, either as a single formulation or concurrently administered, to favorably treat psychiatric and neurological disorders such as depression, schizophrenia, bipolar disorder, attention deficit hyperactivity disorder, Alzheimer's disease and others.

16. The formulation of claim 8, wherein the formulation is in a form for topical use as a component of the formulation for sunscreen lotions, facial and skin care lotions, cleansers, ointments, cosmetics, or scalp, foot, and hair care products, including hair growth.

17. A method of reducing triglycerides in a human in need thereof, the human having a fasting triglyceride level from ≧200 mg/dl to 1500 mg/dl, by administering a formulation of claim 10.

18. The method of claim 17, wherein the R of Formula (I) is EPA, and the dose is from about 0.5 to about 5 g/day in 1-4 doses/day.

19. A method of treating Type 2 diabetes in a human, having Type 2 diabetes defined by fasting plasma glucose of ≧7.0 mmol/l (126 mg/dl) and/or glycated hemoglobin (HbA_{1c}) of greater than 6.5%, by administration of an effective amount of a formulation of claim 10 to the human in need of such treatment.

20. The method of claim 19, wherein the R of Formula (I) is EPA, and the effective dose is from about 0.5 to about 5 g/day in 1-4 doses/day.

21. The method of claim 19 for the treatment of Type 2 diabetes, wherein the formulation is used in concert with metformin hydrochloride, about 500 mg, as 2 doses/day, administered either as a single formulation or concurrently administered.

22. (canceled)

23. (canceled)

24. The formulation of claim 12, wherein the formulation is used in a dose of 50-1000 mg/day, in combination with commonly prescribed medications in their usual dose or reduced dose, either as a single formulation or concurrently administered, to treat attention deficit hyperactivity disorder in children 6-12 years and teenagers.

25. The of claim 24, wherein the medications are atomoxetine hydrochloride (Concerta®), methylphenidate (Ritalin®), dextroamphetamine plus amphetamines (Adderall®), or lisdexamfetamine (Vynanse®).
